(19) United States
(12) Patent Application Publication
(10) Pub. No.: US 2022/0301678 A1
(43) Pub. Date: Sep. 22, 2022

(54) SYSTEMS AND METHODS FOR MONITORING ADHERENCE TO SUBSTANCE REPLACEMENT THERAPY USING A SUBSTANCE DELIVERY DEVICE

(71) Applicant: ResMed Inc., San Diego, CA (US)

(72) Inventors: Joy Zhang, Sydney (AU); Rodney Sherwin Elias, Sydney (AU); Svetlana Poonka, San Diego, CA (US); Sarah Megan Dench, San Diego, CA (US); Abhishek Reddy Yerragonda, San Diego, CA (US)

(21) Appl. No.: 17/636,459

(22) PCT Filed: Aug. 20, 2020
(86) PCT No.: PCT/US2020/047234

§ 371 Date: Feb. 18, 2022

Related U.S. Application Data
(60) Provisional application No. 62/890,883, filed on Aug. 23, 2019.

(51) Int. Cl. G16H 20/13; A61B 5/00; G16H 50/30; G16H 40/63

(52) U.S. Cl. CPC G16H 20/13; A61B 5/746; A61B 5/743; A61B 5/7275; G16H 50/30; G16H 40/63

(57) ABSTRACT

A system includes a substance delivery device, a sensor, and a control system. The substance delivery device is configured to deliver a substance. The sensor is configured to generate data associated with the delivery of the substance. The control system is configured to accumulate the generated data including historical data and current data. The control system is configured to determine that the user is currently consuming the substance based at least in part on an analysis of the current data. Responsive to the determination that the user is currently consuming the substance, the control system is configured to determine a current craving score for the user based at least in part on the current data, the historical data, or both. Based at least in part on the current craving score, the control system is configured to determine an intervention.

### CROSS-REFERENCE TO RELATED APPLICATION(S)

This application claims priority to U.S. Provisional Patent Application No. 62/890,883, filed Aug. 23, 2019, which is hereby incorporated by reference herein in its entirety.

### TECHNICAL FIELD

The present disclosure relates to monitoring substance adherence therapies and more specifically to systems and methods for determining a substance usage of a user and tailoring interventions based on the determined substance usage.

### BACKGROUND

Substance abuse can lead to overall societal harm, and not just potential harm to an individual consuming the substance. For example, overall societal impact of substance abuse can be represented as overall economic costs, which can include health-related costs, productivity losses, and non-health direct expenditures. Health-related costs include hospitalization costs, emergency room costs, costs for health specialists that monitor the individual both at in-patient and out-patient facilities, and so on. Productivity losses include losses due to premature death, substance-abuse related illnesses, incarceration, and so on. Non-health direct expenditures include costs associated with the criminal justice system, crime victim costs, and so on.

With access to recreational and prescription substances, such as, cannabis, alcohol, psilocybin, nicotine, opioids, amphetamines, 3,4-methylenedioxy-methamphetamine (MDMA), and so on, a need exists for systems and methods for tracking individual consumption and taking measures to prevent and/or reduce the risk of substance abuse. Traditional methods rely on self-reporting data which adds burden to the individual, lack personalization which leads to ineffective support of the individual, and so on. The present disclosure provides systems and methods that address drawbacks of traditional methods and also provide additional benefits.

### SUMMARY

According to some implementations of the disclosure, a method includes receiving data associated with a delivery of a substance to a user. The data includes historical data and current data. The user is determined to be currently consuming the substance based at least in part on an analysis of the current data. Responsive to the determination that the user is currently consuming the substance, a current craving score for the user is determined based at least in part on the current data, the historical data, or both. An intervention is determined based at least in part on the current craving score.

According to some implementations of the present disclosure, a method includes receiving current data and historical data associated with a substance consumption triggering event. The substance consumption triggering event is an event that occurs within a predetermined amount of time before a substance delivery device delivers an amount of a substance to a user. The current data is analyzed to determine whether the substance consumption triggering event is present. A likelihood that the user will consume the substance via the substance delivery device within the predetermined amount of time is determined, based at least in part on (i) the determined substance consumption triggering event being present, (ii) the current data, (iii) the historical data, or (iv) any combination of (i), (ii), and (iii).

According to some implementations of the present disclosure, a method includes receiving data associated with a delivery of a substance to a user. The data includes historical data and current data. A machine learning algorithm is trained with the historical data, such that the machine learning algorithm is configured to (i) receive as an input the current data and (ii) determine as an output a predicted time or a predicted location that the user will consume the substance via a substance delivery device.

According to some implementations of the present disclosure, a method includes receiving data associated with one or more substance consumption triggering events. The data includes current data and historical data. The substance consumption triggering events are events that occur within a predetermined amount of time before a substance delivery device delivers an amount of the substance to the user. By analyzing the current data, one of the one or more substance consumption triggering events is determined to be present. Responsive to the determination that one of the one or more substance consumption triggering events is present, a likelihood that the user will consume the substance via the substance delivery device within the predetermined amount of time is determined, based at least in part on (i) the determined substance consumption triggering event being present, (ii) the current data, (iii) the historical data, or (iv) any combination of (i), (ii), and (iii). Responsive to the determined likelihood satisfying a threshold, a current adherence score for the user is determined, based at least in part on the current data, the historical data, or both, the current adherence score indicating an overall adherence level of the user to a substance replacement therapy. Based at least in part on the current adherence score, an intervention is determined.

According to some implementations of the disclosure, a system includes a substance delivery device, a sensor, a memory, and a control system. The substance delivery device is configured to deliver a substance to a user. The sensor is configured to generate data associated with the delivery of the substance to the user. The memory is configured to store machine-readable instructions. The control system includes one or more processors configured to execute the machine-readable instructions to: (i) accumulate the generated data, the generated data including historical data and current data; (ii) determine that the user is currently consuming the substance based at least in part on an analysis of the current data; (iii) responsive to the determination that the user is currently consuming the substance, determine a current craving score for the user based at least in part on the current data, the historical data, or both; and (iv) based at least in part on the current craving score, determine an intervention.

According to some implementations of the disclosure, a system includes a substance delivery device, a sensor, a memory, and a control system. The substance delivery device is configured to deliver a substance to a user. The sensor is configured to generate current data and historical data associated with a substance consumption triggering event. The substance consumption triggering event is an event that occurs within a predetermined time before the substance delivery device delivers an amount of the substance to the user. The memory stores machine-readable instructions. The control system includes one or more processors configured to execute the machine-readable instructions to: analyze the current data to determine whether the substance consumption triggering event is present; and determine a likelihood that the user will consume the substance via the substance delivery device within the predetermined amount of time based at least in part on (i) the determined substance consumption triggering event being present, (ii) the current data, (iii) the historical data, or (iv) any combination of (i), (ii), and (iii).

According to some implementations of the disclosure, a system includes a substance delivery device, a sensor, a memory, and a control system. The substance delivery device is configured to deliver a substance to a user. The sensor is configured to generate data associated with the delivery of the substance. The memory stores machine-readable instructions. The control system includes one or more processors configured to execute the machine-readable instructions to: accumulate the data, the data including historical data and current data; and train a machine learning algorithm with the historical data such that the machine learning algorithm is configured to (i) receive as an input the current data and (ii) determine as an output a predicted time or a predicted location that the user will consume the substance via the substance delivery device.

According to some implementations of the disclosure, a system includes a substance delivery device, a sensor, a memory, and a control system. The substance delivery device is configured to deliver a substance to a user. The sensor is configured to generate data associated with one or more substance consumption triggering events. The data includes current data and historical data. The substance consumption triggering events are events that occur within a predetermined time before the substance delivery device delivers an amount of the substance to the user. The memory stores machine-readable instructions. The control system includes one or more processors configured to execute the machine-readable instructions to: determine, by analyzing the current data, that one of the one or more substance consumption triggering events is present; responsive to the determination that one of the one or more substance consumption triggering events is present, determine a likelihood that the user will consume the substance via the substance delivery device within the predetermined amount of time based at least in part on (i) the determined substance consumption triggering event being present, (ii) the current data, (iii) the historical data, or (iv) any combination of (i), (ii), and (iii); responsive to the determined likelihood satisfying a threshold, determine a current adherence score for the user based at least in part on the current data, the historical data, or both, the current adherence score indicating an overall adherence level of the user to a substance replacement therapy; and based at least in part on the current adherence score, determine an intervention.

According to some implementations of the disclosure, a system includes a substance delivery device, a sensor, a memory, and a control system. The substance delivery device is configured to deliver a substance to a user. The sensor is configured to generate data associated with the delivery of the substance. The memory stores machine-readable instructions. The control system includes one or more processors configured to execute the machine-readable instructions to: accumulate the data, the data including historical data and current data; analyze the current data to determine whether a substance consumption triggering event is present, the substance consumption triggering event being an event that occurs within a predetermined time before the substance delivery device delivers an amount of the substance to the user; and train a machine learning algorithm with the historical data such that the machine learning algorithm is configured to (i) receive as an input the substance consumption triggering event, at least a portion of the current data, or both and (ii) determine as an output a predicted time or a predicted location that the user will consume the substance via the substance delivery device.

The foregoing and additional aspects and implementations of the present disclosure will be apparent to those of ordinary skill in the art in view of the detailed description of various embodiments and/or implementations, which is made with reference to the drawings, a brief description of which is provided next.

### BRIEF DESCRIPTION OF THE DRAWINGS

The foregoing and other advantages of the present disclosure will become apparent upon reading the following detailed description and upon reference to the drawings.

FIG. 1 illustrates an environment for monitoring adherence to a substance replacement therapy, according to some implementations of the present disclosure;

FIG. 2 is a block diagram of a system for monitoring adherence to a substance replacement therapy, according to some implementations of the present disclosure;

FIG. 3 is a flow diagram for determining an intervention based on a detected substance consumption, according to some implementations of the present disclosure;

FIG. 4 is a flow diagram for determining a likelihood of consumption of a substance based on a substance consumption triggering event, according to some implementations of the present disclosure.

FIG. 5 is a flow diagram for determining an intervention based on a substance consumption triggering event and a likelihood of consumption of a substance, according to some implementations of the present disclosure.

While the present disclosure is susceptible to various modifications and alternative forms, specific implementations have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DETAILED DESCRIPTION

Traditional standard of care for individuals reliant on a substance involves a combination of pharmacotherapy and behavior change support. For example, individuals reliant on smoking are provided with pharmacotherapies including medication (e.g., varenicline and buproprion) and nicotine replacement therapy (NRT). NRT can include patches, gum, lozenge, sprays, inhalators, and so on. NRT helps to reduce cravings and withdrawal symptoms associated with quitting smoking. Behavioral change support can take a form of face-to-face or telephonic support, individual and group counseling sessions, or digital support (e.g., SMS programs, online programs, mobile apps, etc., or any combination thereof).

The present disclosure provides several advantages over the traditional standard of care. In some implementations, the present disclosure provides a system for predicting cravings and/or triggers by monitoring smokers' behaviors. Wearables (e.g., smart watches, smart clothing, smart jewelry, etc.) and other sensors can facilitate monitoring smokers' behaviors. Wearables can be used to track smoking gestures. Smart cigarette cases or lighters or packaging, and buttons that are manually activated by a smoker can be used to automatically self-report a craving. Data is collected from these sources and/or other sources to monitor cigarette consumption and/or consumption of one or more substances (e.g., nicotine, cannabis, etc.) via one or more devices (e.g., vaporizers, inhalers, e-cigarettes, gum, patches, vape pens, etc., or any combination thereof) and/or anticipate consumption (e.g., smoking) episodes and/or cravings in order to drive intervention and support in, for example, real-time and/or substantially real-time.

Some implementations of the present disclosure address poor adherence to nicotine replacement therapy by smokers, which can lead to poor success with a smoking cessation treatment regime. Poor adherence is addressed by incorporating different sensors into the system, thus, the system does not need to rely entirely on self-reporting by the user, as in traditional standard of care. Self-reporting alone may not be reliable and can add additional burden to the smoker who may be trying to quit smoking and/or reduce consumption of one or more substances.

Some implementations of the present disclosure address lack of personalization of some forms of behavioral support therapy. Behavioral support therapy can have some drawbacks, for example, time-limited group therapy, online programs, and mobile apps may not always be available or customizable. This lack of availability and customization can lead to deliverance of support and advice that is not tailored or useful for the smoker.

Furthermore, due to stigma and/or inconvenience, the smoker/consumer may have poor access to behavioral support. This poor access may lead to the smoker attempting to quit without professional help and/or without an optimal standard of care. It is noted that smoking is provided as an example herein, but some or all of the implementations of the present disclosure can be used in other contexts for one or more other substances. For example, the other substances can include cannabis, alcohol, psilocybin, nicotine, opioids, amphetamines, MDMA, etc., or any combination thereof.

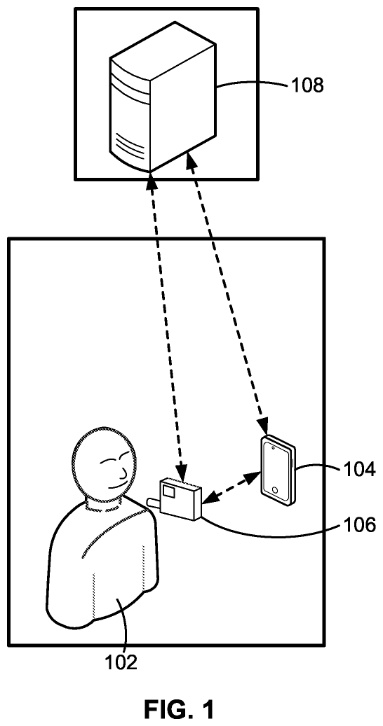

Referring to FIG. 1, an environment for monitoring adherence to a substance replacement therapy is illustrated, according to some implementations of the present disclosure. In a first location, a user 102 (e.g., a consumer of a substance) has a smart inhaler device 106 that is in communication with a mobile phone 104 of the user 102. The smart inhaler device 106 and/or the mobile phone 104 are in communication with a remote server 108 via one or more networks (e.g., a cloud network, the Internet, one or more WANs, one or more LANs, etc., or any combination thereof).

The smart inhaler device 106 delivers a substance to the user 102. The substance can be nicotine, cannabis, alcohol, etc. For example, the smart inhaler device 106 can deliver aerosolized and/or nebulized nicotine through a porous membrane, dry powder, a metered dose inhaler (MDI), other technology, or any combination thereof. The smart inhaler device 106 can produce an optimized nicotine particle size and shape such that the nicotine/substance can to reach the lungs of the user 102 and be readily absorbed (e.g., in a fast acting manner).

The smart inhaler device 106 can provide information about the substance to the mobile phone 104 and/or the remote server 108. The information provided can include a type of substance consumed, an amount of the substance delivered (e.g., for one use), a time of use of the smart inhaler device 106, a geolocation of the smart inhaler 106 during the use, etc. The smart inhaler device 106 can include a button that the user 102 pushes to cause the delivery of the substance to the user 102. The button can also activate an embedded sensor to collect information. The mobile phone 104 can relay the information and/or a portion thereof to the remote server 108 for processing data associated with substance use of the user 102.

The remote server 108 can be a cloud server that supports a mobile client application running on the mobile phone 104. The remote server 108 can be connected to a myriad other users and can store data regarding substance use and/or substance therapy regimens of the other users. The remote server 108 can leverage data from the other users and information from the mobile phone 104 to monitor adherence of a substance replacement therapy and determine interventions for the user 102 and/or for any of the other users. For example, the remote server 108 can push messages to the mobile phone 104 to encourage the user 102 and/or to ask the user 102 to refrain from using the smart inhaler device 106 and/or the like.

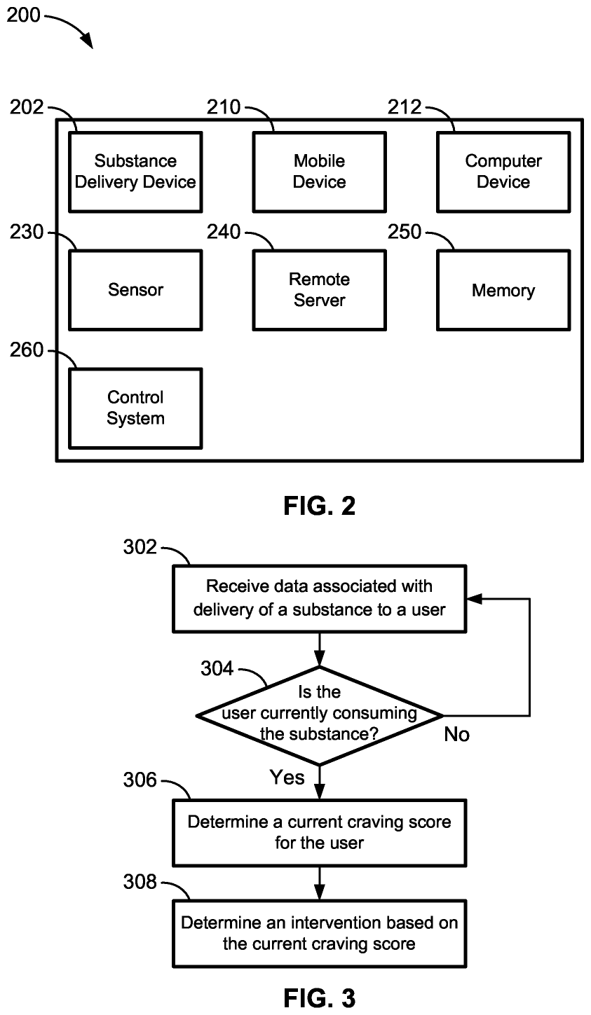

Referring to FIG. 2, a block diagram of a system 200 for monitoring adherence to a substance replacement therapy is shown according to some implementations of the present disclosure. To simplify discussion, the singular form will be used for all components identified in FIG. 2 when appropriate, but the use of the singular does not limit the discussion to only one of each such component.

The system 200 includes a substance delivery device 202. The substance delivery device 202 is configured to deliver a substance to a user (e.g., the smart inhaler device 106 is a substance delivery device configured to deliver a substance to the user 102). Other examples of the substance delivery device 202 include an inhalation device, a metered dose inhaler, a nebulizer device, an aerosol device, a smart patch, a spray device, a vaporizer, an electronic cigarette, an electronic nicotine delivery system, etc.

The system 200 can further include a mobile device 210. The mobile device 210 can be a smart phone (e.g., the mobile phone 104), a laptop device, a tablet device, a smart watch, etc., or any combination thereof. The mobile device 210 can run one or more client applications that facilitate pairing the mobile device 210 to the substance delivery device 202. The mobile device 210 can retrieve substance usage data from the substance delivery device 202. The mobile device 210 can provide settings for configuring the substance delivery device 202. These settings may include an individual use dosage of the substance delivered by the substance delivery device 202, a daily dosage, a weekly dosage, etc.

The system 200 can further include a computer device 212. The computer device 212 can serve a similar function as the mobile device 210. Examples of the computer device 212 include a desktop computer, a smart television, etc., or any combination thereof. The computer device 212 can receive messages relating to substance adherence that are intended for the user. For example, when the user is at work, the computer device 212 can be a work desktop computer. The user can receive, for example, via an email service, the messages relating to substance adherence. The computer device 212 can also include devices of other users, e.g., a caregiver of the user. Examples of caregivers include family members, nurses, doctors, counselors, physicians, friend, anyone involved in a treatment plan of the user, or any combination thereof. The computer device 212 of a caregiver can provide settings for an amount or dosage of substance to be provided by the substance delivery device 202.

The system 200 further includes a sensor 230. The sensor 230 can be configured to generate data associated with the delivery of the substance to the user. The sensor 230 can be a wearable device coupled (e.g., wirelessly and/or wired) to the mobile device 210, the computer device 212, and/or the substance delivery device 202. The sensor 230 includes one or more clocks, one or more global positioning service (GPS) receivers, or one or more indoor positioning systems (e.g., indoor positioning systems using Bluetooth, Wi-Fi, or some other wireless protocol), one or more photoplethysmography (PPG) sensors, one or more gas sensors, one or more oxygen saturation sensors, one or more pressure sensors, one or more flow rate sensors, one or more sweat sensors, one or more cameras, one or more microphones, one or more heart rate sensors, one or more magnetic field sensors, one or more accelerometers, one or more optical sensors, one or more impedance sensors, etc. or any combination thereof.

The sensor 230 can generate data that includes an amount of the substance consumed during one or more consumption sessions, a current rate of consumption of the amount of the substance, one or more time intervals between consumptions of the substance, an average amount of the substance consumed within a predefined period, a geolocation of the user during a consumption session, or any combination thereof. In some implementations, the sensor 230 is and/or includes a flow meter, a gas sensor, and a clock located on the substance delivery device 202. The flow meter, the gas sensor, and the clock provide a time when the user consumed the substance and a rate at which the substance flowed through the substance delivery device 202, and a concentration of the substance that flowed through the substance delivery device 202.

In some implementations, the sensor 230 is and/or includes a GPS or another positioning system that generates data that is indicative of current location and/or a current time of day of the user 102, of the substance delivery device 202, of the mobile device 210, of the computer device 212, or any combination thereof. The sensor 230 can leverage geo-positioning/geolocation data to aid in identification of business names, business footprints, etc., such that location data obtained can be correlated and meaningful to the user (e.g., helpful in determining possible triggers for consumption by the user). The sensor 230 can not only include gas sensors for sensing the substance, but can have the gas sensors measure a carbon monoxide level exhaled by the user.

The sensor 230 can further generate data which includes heart rate data, current stress data (e.g., estimated via heart rate variability), blood pressure data, or any combination thereof. The sensor 230 can include microphones, PPG sensors, electrocardiogram (ECG) sensors for measuring the heart rate and blood pressure of the user. The sensor 230 can include one or more wearable devices coupled to the user and/or can be located on and/or in the substance delivery device 202, the mobile device 210, the computer device 212, or any combination thereof.

The sensor 230 can be calibrated and/or configured by the mobile device 210, the computer device 212, or both. In some implementations, the sensor 230 can access and utilize self-reported data of the user via the pairing with the mobile device 210. For example, the mobile device 210 can have access to self-reported craving data. The self-reported craving data can include a feeling of a craving of the substance on a predetermined scale. For example, the mobile device 210 can prompt the user for a feeling of craving on a scale from 1 to 10 with 10 being a highest intensity of craving and 1 being a lowest intensity of craving.

The mobile device 210 can also facilitate self-reporting of mood data. For example, the mobile device 210 can prompt the user to provide her mood from a predefined selection of moods. For example, the mobile device 210 can prompt the user to indicate whether she is happy, neutral, sad, anxious, disengaged, etc. The mobile device 210 can further probe a level of intensity of the mood. For example, the mobile device 210 can ask how sad the user is on a scale from 1 to 5 where 5 indicates a highest level of sadness and 1 indicates a lowest level of sadness.

The mobile device 210 can also facilitate self-reporting of trigger data. For example, the user can indicate that traveling from work to home or from home to work usually induces and/or triggers a craving to ingest the substance from the substance delivery device 202. The user can also indicate that getting to her home location is also an event that induces and/or triggers a craving to ingest the substance. Other examples of triggers can include the user being at a work location of the user, a time of day, the user being at a bar, the user being in a school, the user being in a restaurant, the user having an elevated heart rate, the user having an increased sweat production, the user being at a known triggering location for other users, the user being around one or more specific persons (e.g., a parent, a sibling, a friend, a teacher, an officer, etc.), the user being in public, the user being alone, the user walking a pet (e.g., a dog), etc. or any combination thereof.

The mobile device 210 can also facilitate self-reporting of consumption episodes/sessions (e.g., recent consumption episodes/sessions). In some implementations, the sensor 230 may not capture a substance consumption event/session. For example, the user may not use the substance delivery device 202 or may use another person's substance delivery device. The sensor 230 being unable to capture data associated with the user using another person's substance delivery device may rely on self-reporting of recent consumption episodes obtained from the mobile device 210.

The mobile device 210 can also facilitate self-reporting of symptoms, withdrawal effects, or both. For example, the sensor 230 may be able to capture heart rate data, sweat data, and so on, but may be unable to capture events like vomiting, nausea, light-headedness, etc. The mobile device 210 can prompt the user to report symptoms or can provide the user with a list of common symptoms or permit the user to enter custom symptoms. The user can either enter their symptoms and/or can select from the provided list.

The system 200 can further include a remote server 240. The remote server 240 can represent one or more cloud computing servers for running server-side applications to support and/or work with the client application on the mobile device 210. The remote server 240 performs calculations, runs machine learning algorithms, and performs statistical analyses to determine trends and substance usage patterns and events of the user. The remote server 240 can determine interventions and push messages to the mobile device 210, the computer device 212, or both, in order to alert or encourage the user. In some implementations, the mobile device 210 and/or the computer device 212 performs some or all of the tasks described for the remote server 240.

The memory 250 can include one or more physically separate memory devices, such that one or more memory devices can be coupled to and/or built into the substance delivery device 202, the mobile device 210, the computer device 212, the remote server 240, a control system 260, and/or one or more external devices wirelessly coupled and/or wired to any component of the system 200, or any combination thereof. The memory 250 acts as a non-transitory computer readable storage medium on which is stored machine-readable instructions that can be executed by the control system 260 and/or one or more other systems. The memory 250 is also able to store (temporarily and/or permanently) the data generated by the sensor 230. In some implementations, the memory 250 includes non-volatile memory, battery powered static RAM, volatile RAM, EEPROM memory, NAND flash memory, or any combination thereof. In some implementations, the memory 250 is a removable form of memory (e.g., a memory card).

Like the memory 250, the control system 260 can be coupled to and or positioned within the substance delivery device 202, the mobile device 210, the computer device 212, the sensor 230, the remote server 240, one or more external devices, or any combination thereof. The control system 260 is coupled to the memory 250 such that the control system 260 is configured to execute the machine-readable instructions stored in the memory 250. The control system 260 can include one or more processors and/or one or more controllers. In some implementations, the one or more processors includes one or more x86 INTEL processors, one or more processors based on ARM® Cortex®-M processor from ARM Holdings such as an STM32 series microcontroller from ST MICROELECTRONIC, or any combination thereof.

In some implementations, the control system 260 is a dedicated electronic circuit. In some implementations, the control system 260 is an application-specific integrated circuit. In some implementations, the control system 260 includes discrete electronic components.

The control system 260 is able to receive input(s) (e.g., signals, generated data, instructions, etc.) from any of the other elements of the system 200 (e.g., the sensors 230, etc.). The control system 260 is able to provide output signal(s) to cause one or more actions to occur in the system 200 (e.g., to cause the remote server 240 to send a message to the mobile device 210 at a specific time, etc.).

While the control system 260 and the memory 250 are described and shown in FIG. 2 as being separate and distinct component of the system 200, in some implementations, the control system 260 and/or the memory 250 are integrated in the substance delivery device 202 and/or the mobile device 210, and/or the computer device 212, and/or the remote server 240. Alternatively, in some implementations, the control system 260 or a portion thereof (e.g., at least one processor of the control system 260) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While the system 200 is shown as including all of the components described above, more or fewer components can be included in a system according to implementations of the present disclosure. For example, a first alternative system includes the control system 260, the memory 350, and at least one of the sensors 230 provided in FIG. 2. As another example, a second alternative system includes the control system 260, the memory 250, the substance delivery device 202, and at least one of the sensors provided in FIG. 2. Thus, various systems can be formed using any portion or portions of the components shown and described herein and/or in combination with one or more other components.

Referring to FIG. 3, a flow diagram illustrating a method for determining an intervention based on a detected substance consumption is shown according to some implementations of the present disclosure. At step 302, the control system 260 receives data associated with delivery of a substance to the user. The received data can be data generated by the sensor 230 and/or data obtained by self-reporting (e.g., via the mobile device 210, via the computer device 212, etc.). The received data can include both historical data and current data. Current data can be defined as data pertaining to a most recent time period, such as, for example, data pertaining to the past three minutes, five minutes, ten minutes, thirty minutes ago, hour, etc. Historical data can be defined as data obtained prior to the most recent time period (which can be defined by the user and/or the system).

At step 304, the control system 260 determines whether the user is currently consuming the substance. The determination can be based at least in part on an analysis of the current data. For example, the sensor 230 providing flow rate data for a release of the substance can indicate that the user is consuming the substance. The sensor 230 measuring an increased heart rate can indicate that the user is consuming the substance. The sensor 230 measuring an increase in exhaled carbon monoxide can indicate that the user is consuming the substance. The mobile device 210, determining that the user usually consumes the substance at a certain time of day, can correlate sweat data, heart rate variability data, and other physiological measurements of the user to determine whether the user is currently consuming the substance.

If the user is not currently consuming the substance, the control system 260 can continue monitoring current data at step 302. If the user is currently consuming the substance, then the control system 260 can determine a current craving score for the user. A craving score according to some implementations is a measure of thirst that the user feels for the substance. The current craving score is a craving score determined within a most recent period. The most recent period can be, for example, within the last three minutes, five minutes, ten minutes, thirty minutes, one hour, and so on.

The current craving score can be determined from the current data, the historical data, or both. The current craving score can also be determined based on previous or historical craving scores. For example, at an initial time period, the control system 260 can determine that the user is consuming the substance, and based on current heart rate variability and current amount of substance consumed, determine the current craving score to be 6 on a scale of 1 to 10. After an hour, the control system 260 can determine that the user is again consuming the substance. Even though current heart rate variability and current amount of substance consumed are similar to the initial time period, the control system 260 can determine that based on the amount of time between substance consumption episodes, the current craving score should be 6.5 instead of 6.

The current craving score can also be determined based on a failed intervention, where a failed intervention is an action provided to the user by the system 200 that failed to prevent, for example, a consumption session, an increase in consumption of the substance, or any combination thereof. Using the previous example, after determining that the current craving score is 6.5, the control system 260 can send a message to the mobile device 210 to inform the user of his current craving score. If the user then consumes the substance within a buffer time (e.g., thirty minutes, an hour, two hours, etc.), the control system 260 can determine for the most recent consumption episode that the craving score should be 7.5. A larger increase can be embedded in the determined score to take into account the failed intervention.

After determining the current craving score, at step 308, the control system 260 can determine an intervention based on the current craving score. In some implementations, the control system 260 can adjust a setting on the substance delivery device 202, the mobile device 210, or both. For example, the control system 260 can adjust an amount of substance to be delivered to the user for a next consumption episode. The amount of substance can be adjusted via a client application running on the mobile device 210 and then relayed to the substance delivery device 202. The amount of substance can be adjusted directly on the substance delivery device 202.

In some implementations, the control system 260 can increase or decrease the amount of the substance delivered to the user based on the current craving score being greater than a previous craving score. For example, the user can self-report via the mobile device 210 that withdrawal symptoms are still present after consuming the substance. The control system 260 can take into account severity of the withdrawal symptoms in determining the current craving score. Based on the effect of the severity of the withdrawal symptoms on the current craving score, the control system 260 can increase the amount of substance delivered to the user. The control system 260 can adjust the setting on the substance delivery device 202 to increase the amount of substance delivered or can configure the substance delivery device 202 to allow multiple doses of the substance to realize the increase in amount.

In some implementations, the control system 260 can adjust a maximum daily amount of the substance to be delivered to the user. For example, when the user is approaching her maximum daily amount, the control system 260 can determine that the current craving score is too high based on a number of hours left in the day. The control system 260 can then increase the maximum daily amount of the substance. In some implementations, the control system 260 can decrease the maximum daily amount of the substance based on the current craving score being low and an expected number of future consumption episodes not depleting the maximum daily amount of the substance.

At step 308, interventions can also include messages sent by the control system 260 to the user via the mobile device 210 (and/or the computer device 212 in some implementations). The control system 260 can have multiple messages stored in the memory 250 to choose from. The messages can be categorized as an encouraging message, an award message, a behavioral strategy message, an educational message, a distracting message, a request for information, or any combination thereof. There can be more than one message under each category.

In some implementations, the control system 260 determines to send a message from a specific category based at least in part on the current craving score. For example, if the current craving score is 40 on a scale of 1 to 100, the control system 260 can select an encouraging message or an award message to send to the user. In another example, if the current craving score is below 60%, 50%, 40%, etc., of a highest possible craving score, the control system 260 can send the encouraging message or the award message. Similarly, the control system 260 can send an educational message, a distracting message, or a request for more information when the current craving score does not satisfy a threshold. The request for more information can be a survey or questionnaire.

In some implementations, the control system 260 determines a category of the message to be sent based on a previous message sent to the mobile device 210. For example, the control system 260 determines that an encouraging message was not previously effective and that the current craving score is higher than a previous craving score after the encouraging message was sent. The control system 260 can then choose to send an award message instead of sending another encouraging message.

In some implementations, the control system 260 does not switch categories but determines that the specific encouraging message may not have been effective. The control system 260 then selects a different encouraging message within the encouraging message category. In some implementations, the control system 260 can suggest alternative messaging based on user preferences. For example, the user can indicate that a category is ineffective, and the control system 260 can use context to determine that the message sent should be a prompt such as taking a different route to work or replacing certain types of food in the user's diet.

In some implementations, the control system 260 determines that stress or heart rate variability is highly correlated with the user using the substance delivery device 202. The control system 260 can then determine the message to be one of combating stress. For example, the control system 260 can provide advice on deep breathing or other methods of reducing stress and anxiety.

In some implementations, the control system 260 can use self-reported preferences stored in a user profile of the user. For example, for some messages sent to the mobile device 210, the control system 260 can include a prompt such that the user can rate or provide feedback and effectiveness of the messages. Based on certain messages being indicated by the user as being ineffective, the control system 260 can select one or more other messages that are not under the ineffective category to send to the mobile device 210. The control system 260 can also augment user preferences with preferences from other users where certain messages indicated by other users as being ineffective are not sent to the mobile device 210.

In some implementations, the control system 260 determines a timing for when to send messages to the mobile device 210 (and/or the computer device 212 in some implementations). The control system 260 can determine to send a message to the mobile device 210 at a predetermined time after determining that the user is no longer consuming the substance. The predetermined time can be five minutes after, ten minutes after, thirty minutes after, and so on. The predetermined time can be based on determining a best time to capture the user's attention.

In some implementations, the control system 260 can determine to send the message to the mobile device 210 while the user is still consuming the substance. The message sent while the user is still consuming can be based on a strategy to interrupt the current substance consumption episode.

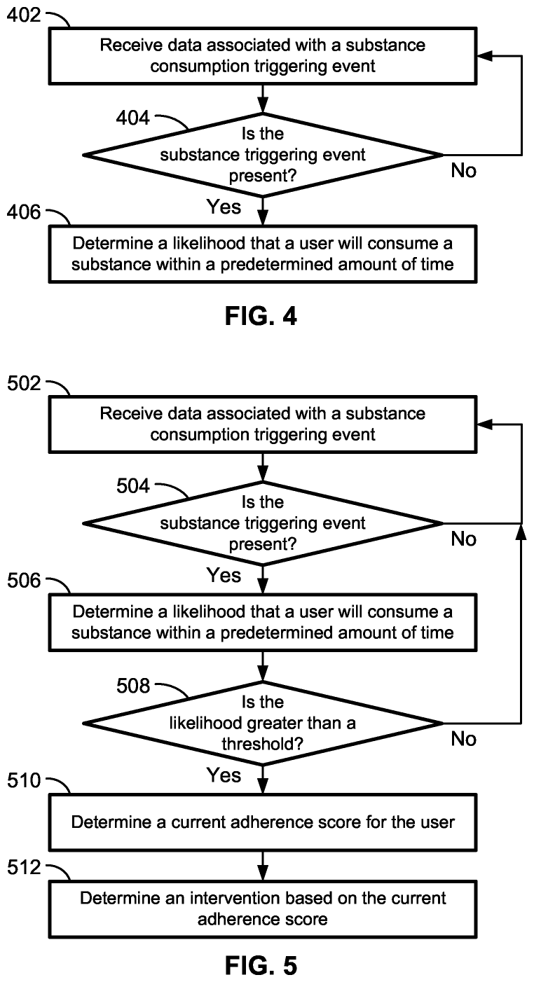

FIG. 3 provided a scenario where a substance consumption episode was underway and a current craving score was determined to guide a determination of an intervention. FIG. 4, on the other hand, tries to anticipate whether a substance consumption episode will occur within a predetermined amount of time. Referring to FIG. 4, a flow diagram for determining a likelihood of consumption of a substance based on a substance consumption triggering event is provided, according to some implementations of the present disclosure. A substance consumption triggering event is an event that occurs within a predetermined time before the substance delivery device 202 delivers an amount of the substance to the user or before the user consumes the substance. The predetermined time can be, for example, within thirty minutes, twenty minutes, ten minutes, five minutes, one minute, thirty seconds, etc.

At step 402, the control system 260 receives data associated with the substance consumption trigger event. Step 402 is similar to step 302, where similar data is collected using the sensor 230 and the mobile device 210 and where the data includes current data and historical data.

At step 404, the control system 260 analyzes the current data to determine whether the substance triggering event is present based on the received data. The substance triggering event can be identified as such by the control system 260 using historical data. For example, the historical data can be used to determine a timeline of events. From the timeline of events, the control system 260 can identify one or more features within the timeline as being one of a plurality of substance consumption triggering events. The features can include a change in location or a specific time of day leading to a substance consumption event.

In some implementations, examples of substance triggering events include (i) the user traveling from a source location to a destination location, (ii) the user arriving or being at a home location of the user, and (iii) the user arriving or being at a work location of the user. The user may be prone to consuming the substance within a predetermined amount of time after reaching a destination, which can be after reaching the user's home, work, school, bar, restaurant, and so on. The user may be prone to consuming the substance if staying at a certain location for a time longer than the predetermined amount of time. For example, if the user is at the user being at the home location of the user for most of the day can be a triggering event. The user may be prone to consuming the substance during transit from one location to another. The control system 260 analyzes the current data to identify location-related triggers for the user.

In some implementations, the substance triggering event can be based on features of the day. For example, the user may be prone to consuming the substance around a same time of the day. The user may consume the substance around breakfast time, lunch time, dinner time, etc. Time of the day can include specific times of the day, e.g., 9 AM, 10 AM, 6 PM, 11 PM, etc.

Determination of the substance triggering event can be based on physiological measurements of the user captured by the sensor 230, the mobile device 210, or both. For example, determination of substance triggering events can include an analysis of heart rate, sweat production, blood pressure, etc. Substance triggering events resulting from the analysis can include the user having an elevated heart rate, the user having an increased sweat production, the user having an elevated blood pressure, the user having a decreased heart rate variability, the user self-reporting a low mood, the user self-reporting anxiety, the user self-reporting a craving, the user self-reporting withdrawal symptoms, or any combination thereof.

In some implementations, substance triggering events from other users can be searched for within the current data. That is, learned patterns and behavior from other users can be leveraged for determining if a particular user is experiencing and/or has experienced a substance triggering event. For example, substance triggering events can include the user being at a known triggering location for other users, or a time of day where other users are commonly triggered. The control system 260 can aggregate data from multiple users to determine common locations which have high instances of usage for the substance delivery device 202.

At step 406, the control system 260 can determine a likelihood that the user will consume the substance via the substance delivery device 202 within the predetermine amount of time. The likelihood can be determined based at least in part on the determined substance triggering event being present, the current data, the historical data, or any combination thereof. For example, based on the determined substance triggering event of step 404, the control system 260 can determine the likelihood by comparing timing information in the current data to timing information in the historical data. The timing information in the historical data can include an average time elapsed between the determined substance triggering event and substance consumption episodes. The timing information in the current data can include time elapsed since the determined substance triggering event occurred in the current data.

In some implementations, the control system 260 only uses a subset of the historical data in determining the likelihood. For example, the subset of the historical data can be windowed by two months, a month, two weeks, a week, three days, a day, etc. That way, the control system 260 can capture a most applicable substance consumption behavior of the user without a longer history that may no longer be applicable skewing the historical data. The windowing of the historical data can be useful because it can provide a period to view the historical data. The period can be used by the control system 260 in determining an average probability that the user will consume the substance within the predetermined time after the determined substance consumption triggering event.

In some implementations, the determined average probability can be taken as the likelihood. The control system 260 can determine the average probability by analyzing the historical data for the period to determine a number of the determined substance consumption triggering event and an occurrence of one or more substance deliveries by the substance delivery device 202. The average probability is then determined as a number of the occurrence of the one or more substance deliveries divided by the number of the plurality of the determined substance consumption triggering event.

In some implementations, the control system 260 can adjust a setting on the substance delivery device 202, the mobile device 210, or both based on the determined likelihood. An adjustment can be the same as, or similar to, step 308 described above in connection with FIG. 3. For example, the control system 260 can increase or decrease a next dosage of the substance delivered by the substance delivery device 202, a total daily dosage of the substance delivered by the substance delivery device 202, or both. The control system 260 can also adjust the next dosage of the substance based on the likelihood, while maintaining the total daily dosage of the substance.

In some implementations, the control system 260 can send messaging to the mobile device 210, the computer device 212, or both, based on the determined likelihood of step 406. The control system 260 can send messaging in a same manner or similar manner as described above in connection with step 308 of FIG. 3. For example, the control system 260 can send messages to the user and/or a caregiver of the user based on the determined likelihood. The control system 260 can determine a category of message to send based on the determined likelihood. The control system 260 can also determine to let the predetermined time elapse without taking an action or causing an intervention.

FIG. 3 introduced using a current craving score to determine an intervention, and FIG. 4 introduced using a substance triggering event to determine a likelihood of consumption. In some implementations, elements of FIGS. 3 and 4 can be combined. For example, in FIG. 3, the current craving score was determined when the user was consuming the substance. In some implementations, the current craving score can be determined even when the user is not currently consuming the substance. That is, the current craving score can be tracked throughout a time period, for example, tracked throughout a day, to obtain a craving trend. The craving trend can be compared with one or more previous days' craving trends. Tracking the current craving score can allow intervening prior to the user consuming the substance once the current craving score satisfies a craving threshold.

In some implementations, the craving score can be estimated for a future time period from the historical data, current data, or both. For example, based on a previous craving trends, a current craving score, current data, historical data, or any combination thereof, the control system 260 can estimate a future craving score. The future craving score can be used by the control system 260 to intervene prior to the user consuming the substance once the future craving score satisfies a threshold. In some implementations, one or more craving scores (e.g., a current craving score, a future craving score, a craving trend, etc.) can be used by the control system 260 to intervene prior to the user consuming the substance.

In addition to methods of determining substance triggering events already discussed in connection with FIG. 4, the control system 260 can train a machine learning algorithm with the historical data. The trained machine learning algorithm or model can then receive as an input the current data and determine as an output a predicted time that the user will consume the substance using the substance delivery device. In some implementations, the trained machine learning algorithm or model can receive the current data and determine as an output a predicted location where the user will consume the substance using the substance delivery device.

In some implementations, the control system 260 can receive the current data and determine whether a substance consumption triggering event is present. The control system 260 can train the machine learning algorithm to receive as an input the substance consumption triggering event and determine as an output a predicted time that the user will consume the substance. The control system 260 can also determine as an output a predicted location for the consumption of the substance.

In some implementations, the control system 260 can train the machine learning algorithm to determine, based on a time of day, a location, or both, that the substance triggering event is the user eating breakfast, the user eating lunch, the user eating dinner, and so on.

In some implementations, the control system 260 can train the machine learning algorithm to determine, based on the user being at a work location of the user and the time of day, that the substance triggering event further includes stress at work, a work break, or specific events related to the user's work environment.

In some implementations, the control system 260 can train the machine learning algorithm to prioritize substance triggering events based on self-reported strengths of craving data received from the mobile device 210. For example, for moments within the historical data where the control system 260 receives self-reporting feelings of craving from the user as craving data, the control system 260 can use intensity information from the craving data to determine a ranking for the substance triggering events.

In some implementations, the control system 260 can train the machine learning algorithm to determine a pattern of usage for the user consuming the substance. Based on the determined pattern of usage, the control system 260 can determine a predicted time that the user will consume the substance or a predicted location where the user will consume the substance.

In some implementations, the control system 260 can train the machine learning algorithm to determine a category of message to send to the user before, during, or after a consumption episode. The category can be similar to, or the same as, one or more categories previously discussed in connection with step 308 of FIG. 3. For example, during a consumption episode/session, the control system 260 can send a request for more information to cause the mobile device 210 and/or an application running on the mobile device 210 to prompt the user to self-report whether the user is eating breakfast, lunch, or dinner. The additional information obtained from the self-reporting can be used in training the machine learning algorithm. In some implementations, training the machine learning algorithm can take about two weeks, one week, five days, etc.

In some implementations, the control system 260 can focus more on higher priority substance triggering events. For example, if meal times are a stronger trigger than changing location, then the control system 260 can deliver more messages and determine more message combinations to send to the user during a meal time than when the user is changing location. Strength of craving reported by the user can be used to prioritize the substance triggering events.

Referring to FIG. 5, a flow diagram for determining an intervention based on a substance consumption triggering event and a likelihood of consumption of a substance is provided, according to some implementations of the present disclosure. At step 502, the control system 260 receives data associated with a substance consumption triggering event. Description for step 502 is similar to, or the same as, step 402 as discussed in connection to FIG. 4 and step 302 as discussed in connection to FIG. 3.

At step 504, the control system 260 determines whether the substance triggering event is present. Step 504 is similar to, or the same as, step 404 as discussed in connection to FIG. 4. If the substance triggering event is not present, then the control system 260 continues monitoring data from the sensor 230, the mobile device 210, or both, at step 502.

If the substance triggering event is present, then at step 506, the control system 260 determines a likelihood that the user will consume a substance delivered by the substance delivery device 202 within a predetermined amount of time. Step 506 is similar to, or the same as, step 406 as discussed in connection to FIG. 4. If the likelihood is less than or equal to a threshold, then the control system 260 continues monitoring data from the sensor 230, the mobile device 210, or both, at step 502.

If the likelihood is greater than the threshold, then at step 510, the control system 260 determines a current adherence score for the user based at least in part on the current data, the historical data, or both. The current adherence score is a measure of how well the user is following a recommended or prescribed substance replacement therapy. For example, the substance replacement therapy can be a nicotine replacement therapy recommended to the user to help the user stop smoking or reduce the user's dependence on nicotine. The substance replacement therapy can be based on a cognitive behavioral therapy or an acceptance and commitment therapy.

The current adherence score can be based on an amount of the substance that the user consumes. For example, the user can consume more than a daily allotted amount which would indicate a low adherence to the substance replacement therapy. In some implementations, an absolute value of a deviation from the daily allotted amount can be divided by the daily allotted amount to determine the adherence score. The closer this value is to zero, the more adherent the user is to the substance replacement therapy. That is, the user consuming a significant amount more or less than the daily amount can indicate a low adherence to the substance replacement therapy.

In some implementations, the substance replacement therapy provides a schedule of consumption. Instead of looking at only the daily allotted amount, the control system 260 can determine whether the user is meeting consumption goals provided in the schedule of consumption. The consumption goals may be provided for periods of half days, one hour, two hours, three hours, and so on. The current adherence score can then take into account adherence score from a most recent period or can be an aggregation of adherence scores from a number of previous periods combined with the most recent period. The aggregation can be determined from an average, a moving average, an exponential moving average, a weighted moving average, a mode, a median, or any other statistical analysis performed on the adherence scores. Weighted statistical methods can include a recency bias where more recent adherence scores are weighted higher than older adherence scores.

At step 512, the control system 260 can determine an intervention based on the current adherence score. Interventions based on the current adherence score can be similar to, or the same as, the interventions discussed in connection to step 308 of FIG. 3. For example, the control system 260 can send a message to the mobile device 210 based on the current adherence score. The control system 260 can determine a category for the message to be sent based on the current adherence score. The control system 260 can adjust a setting on the mobile device 210, the substance delivery device 202, or both, based on the current adherence score.

In some implementations, the control system 260 can send more than one message to the mobile device 210. For example, the control system 260 can send a first support message to the mobile device 210 and monitor whether the first support message was successful in preventing a consumption event. Based on the first support message being unsuccessful, the control system 260 can send a second support message to the mobile device 210. In some implementations, based on the first support message being successful, the control system 260 can send another support message different from the second support message to the mobile device 210. The different support message, the second support message, and/or the first support message can be messages from a same category, from different categories, or a combination thereof.

Some implementations of the present disclosure provide several advantages over traditional standards of care when used for adherence to nicotine replacement therapy. For example, a user who regularly uses her inhaler at 9 AM can receive supportive messaging to encourage the use of the inhaler over a cigarette. That way, consumption episodes/sessions and events can be automatically captured rather than relying on self-reporting from the user. In another example, a user who regularly uses her inhaler at work can receive tips on how to distract herself from craving a cigarette/substance or can receive tips to keep her hands busy. Keeping her hands busy can prevent her from using the inhaler or another nicotine consumption device.

In some implementations, the intervention can be selected from a database of messages based on a preassigned program (e.g., a nicotine replacement therapy). The messages can then be optimized over a time period by analyzing the response of the user messages delivered over the time period. For example, the control system 260 can determine that the user is unable to successfully reduce his dosage as recommended in the nicotine replacement therapy. The control system 260 can then provide a message to the user asking whether the user wishes to wean off nicotine at a slower rate over a longer time period. The control system 260 can adjust the nicotine replacement therapy for the user to improve the user's adherence to the therapy.

In some implementations, the control system 260 can adjust dosage of nicotine delivered through an inhaler based on the nicotine replacement therapy. For example, dosage of nicotine delivered through the inhaler can be gradually decreased over time during a withdrawal schedule. The control system 260 can adjust the inhaler to not provide any more nicotine once a daily limit is reached.

In some implementations, the sensor 230 is included on a nicotine inhaler and/or on the mobile device 210 to determine a GPS location of the user when using the inhaler for a consumption episode/session. The sensor 230 can also provide a time of day of the consumption episode/session. The sensor 230 can also provide a number of puffs of the inhaler during the consumption episode/session.

In some implementations, the control system 260 can develop a withdrawal schedule based on user input (e.g., the user wanting to quit smoking in 12 weeks). The withdrawal schedule developed can include the following parameters: (i) a period of time for quitting, (ii) an amount of nicotine for each day of the period, and/or (iii) a number of nicotine consumption sessions for each day of the period. The parameters are aimed at aiding the user quit nicotine within the desired time frame. The control system 260 can train a machine learning algorithm or model to analyze data from the sensor 230 and/or the mobile device 210, and modify the withdrawal schedule based on an output of the machine learning algorithm or model.

Alterations or modifications to the withdrawal schedule can include (i) increasing or decreasing the period of time for quitting, (ii) increasing or decreasing a number of nicotine consumption sessions for one or more days within the period of time for quitting, (iii) increasing or decreasing an amount of nicotine for one or more days within the period of time for quitting, or any combination of (i), (ii), and (iii).

The inhaler can be configured to be usable according to the withdrawal schedule. For example, when an allotted amount of nicotine and/or an allotted number of consumption sessions are reached for a given day, the inhaler shuts down and/or is otherwise unusable for the rest of the day. In some implementations, the inhaler can include an override feature that permits the user to exceed the allotted amount of nicotine and/or the allotted number of consumption sessions for the given day. The override feature can be configured to be actuated by a caregiver and not by the user associated with the withdrawal schedule. For example, a doctor of the user can send an override message to the control system 260 using the computer device 212. The control system 260 can then send an override signal to the substance delivery device 202 (i.e., the inhaler).

While the present disclosure has been described with reference to one or more particular implementations, those skilled in the art will recognize that many changes may be made thereto without departing from the spirit and scope of the present disclosure. Each of these embodiments and implementations and obvious variations thereof is contemplated as falling within the spirit and scope of the present disclosure, which is set forth in the claims that follow.

1-66. (canceled)

67. A system comprising:
a substance delivery device configured to deliver a substance to a user;
a sensor configured to generate data associated with the delivery of the substance to the user;
a memory storing machine-readable instructions; and
a control system including one or more processors configured to execute the machine-readable instructions to:
accumulate the generated data, the generated data including historical data and current data;
determine that the user is currently consuming the substance based at least in part on an analysis of the current data;
responsive to the determination that the user is currently consuming the substance, determine a current craving score for the user based at least in part on the current data, the historical data, or both; and
based at least in part on the current craving score, determine an intervention.

68. The system of claim 67, wherein the control system is further configured to execute the machine-readable instructions to, based at least in part on the current craving score, adjust a setting on (i) the substance delivery device, (ii) a mobile device of the user, or (iii) both.

69. The system of claim 68, wherein the setting includes an amount of the substance delivered to the user, a maximum daily amount of the substance to be delivered to the user, or both.

70. The system of claim 69, wherein the control system is further configured to execute the machine-readable instructions to reduce the amount of the substance delivered to the user based on the maximum daily amount of the substance to be delivered to the user.

71. The system of claim 69, wherein the control system is further configured to execute the machine-readable instructions to increase the amount of the substance delivered to the user based on (i) a determination that the current craving score is greater than a previous craving score, (ii) a self-reported feeling of a craving, (iii) a self-reported withdrawal symptom, or (iv) any combination thereof.

72. The system of claim 71, wherein the control system is further configured to execute the machine-readable instructions to increase the amount of the substance delivered to the user by delivering the increased amount of the substance in multiple doses.

73. The system of claim 67, wherein the control system is further configured to execute the machine-readable instructions to, based on a failed intervention, determine that the current craving score is greater than a previous craving score, the failed intervention being an action provided to the user that failed to prevent an increase in a daily consumption of the substance.

74. The system of claim 67, wherein the current data includes a current amount of the substance consumed, a current rate of consumption of the amount of the substance, one or more time intervals between consumption of the substance, an average amount of the substance consumed within a predefined period, current heart rate data, current stress data, heart rate variability data, current blood pressure data, self-reporting a current craving data, self-reporting current mood data, self-reporting current trigger data, self-reporting recent consumption episode data, self-reporting symptom data, a current location, a current time of day, current carbon monoxide data, or any combination thereof.

75. The system of claim 67, wherein the historical data includes one or more historical amounts of the substance consumed by the user, one or more historical rates of consumption of the one or more historical amounts of the substance, one or more historical time intervals between consumption of the substance, an average amount of the substance consumed within a historical predefined period, historical heart rate data, historical stress data, historical heart rate variability data, historical blood pressure data, one or more historical self-reported craving data, one or more historical self-reported trigger data, one or more historical self-reported consumption episode data, one or more historical self-reported symptom data, one or more historical locations, one or more historical times of a day, historical carbon monoxide data, or any combination thereof.

76. The system of any one of claim 67, wherein the intervention includes sending a message to a mobile device of the user, sending a message to a caregiver device associated with a caregiver of the user, or both.

77. (canceled)

78. The system of claim 76, wherein the control system is further configured to execute the machine-readable instructions to determine a category of the message sent to the mobile device based at least in part on (i) the current craving score, (ii) a previous message category of a previous message sent to the mobile device, or both (i) and (ii),
wherein the category includes one or more of an encouraging message, an award message, a behavioral strategy message, an educational message, a distracting message, a request for information, or any combination thereof.

79-88. (canceled)

89. The system of claim 67, wherein the substance delivery device includes an inhalation device, a metered dose inhaler, a nebulizer device, an aerosol device, a smart patch, a spray device, a vaporizer, an electronic cigarette, an electronic nicotine delivery system, or any combination thereof.

90. A system comprising:
a substance delivery device configured to deliver a substance to a user;
a sensor configured to generate current data and historical data associated with a substance consumption triggering event, the substance consumption triggering event being an event that occurs within a predetermined time before the substance delivery device delivers an amount of the substance to the user;
a memory storing machine-readable instructions; and
a control system including one or more processors configured to execute the machine-readable instructions to:
analyze the current data to determine whether the substance consumption triggering event is present; and
determine a likelihood that the user will consume the substance via the substance delivery device within the predetermined amount of time based at least in part on (i) the determined substance consumption triggering event being present, (ii) the current data, (iii) the historical data, or (iv) any combination of (i), (ii), and (iii)
based on the determined likelihood, perform an intervention.

91. The system of claim 90, wherein the intervention includes:
adjusting a setting on (i) the substance delivery device, (ii) a mobile device of the user, or (iii) both (i) and (ii), or
sending a message to (iv) a mobile device of the user, (v) a caregiver device associated with a caregiver of the user, or (vi) both (iv) and (v).

92. The system of claim 91, wherein the setting includes a next dosage of the substance delivered by the substance delivery device, a total daily dosage of the substance delivered by the substance delivery device, or both.

93. The system of claim 92, wherein the control system is further configured to execute the machine-readable instructions to (i) reduce the next dosage of the substance based on the determined likelihood, or (ii) increase the next dosage of the substance based on the determined likelihood.

94. (canceled)

95. The system of claim 92, wherein the control system is further configured to execute the machine-readable instructions to adjust the next dosage of the substance based on the determined likelihood while maintaining the total daily dosage of the substance.

96-102. (canceled)

103. The system of claim 90, wherein the control system is further configured to execute the machine-readable instructions to (i) determine, using the historical data, a timeline of events, and (ii) identify one or more features within the timeline of events as being one of a plurality of substance consumption triggering events.

104. The system of claim 103, wherein the timeline of events indicates an occurrence of one or more location changes prior to an occurrence of one or more substance consumption events, and wherein the control system is further configured to execute the machine-readable instructions to identify the one or more location changes as one of the plurality of substance consumption triggering events.

105. The system of claim 103, wherein the events included in the timeline of events include one or more substance consumption events, one or more substance consumption triggering events, one or more non-substance consumption triggering events, one or more location changes, or any combination thereof.

106. The system of claim 103, wherein the timeline of events indicates an occurrence of one or more of a plurality of substance consumption events occurring within a time threshold of a specific time of day, and wherein the control system is further configured to execute the machine-readable instructions to identify the specific time of day as one of the plurality of substance consumption triggering events.

107. The system of claim 90, wherein the control system is further configured to execute the machine-readable instructions to determine the likelihood by comparing timing information in the current data to timing information in the historical data based on the determined substance consumption triggering event.

108. The system of claim 90, wherein the control system is further configured to execute the machine-readable instructions to determine the likelihood by comparing timing information and location information in the current data to timing information and location information in the historical data based on the determined substance consumption triggering event.

109-121. (canceled)

122. A system comprising:
a substance delivery device configured to deliver a substance to a user;
a sensor configured to generate data associated with one or more substance consumption triggering events, the data including current data and historical data, the substance consumption triggering events being events that occur within a predetermined time before the substance delivery device delivers an amount of the substance to the user;
a memory storing machine-readable instructions; and
a control system including one or more processors configured to execute the machine-readable instructions to:
determine, by analyzing the current data, that one of the one or more substance consumption triggering events is present;
responsive to the determination that one of the one or more substance consumption triggering events is present, determine a likelihood that the user will consume the substance via the substance delivery device within the predetermined amount of time based at least in part on (i) the determined substance consumption triggering event being present, (ii) the current data, (iii) the historical data, or (iv) any combination of (i), (ii), and (iii);
responsive to the determined likelihood satisfying a threshold, determine a current adherence score for the user based at least in part on the current data, the historical data, or both, the current adherence score indicating an overall adherence level of the user to a substance replacement therapy; and
based at least in part on the current adherence score, determine an intervention.

123-124. (canceled)

125. The system of claim 122, wherein the intervention includes sending a message to a mobile device of the user, sending a message to a caregiver device associated with a caregiver of the user, or both.

126. (canceled)

127. The system of claim 125, wherein the control system is further configured to determine a category of the message sent to the mobile device based at least in part on the current adherence score, the category including one or more of an encouraging message, an award message, a behavioral strategy message, an educational message, or a distracting message.

128. The system of claim 127, wherein the distracting message, the encouraging message, the award message, or a combination thereof are sent to the mobile device in response to the determined current adherence score satisfying an adherence threshold.

129. The system of claim 127, wherein the behavioral strategy message, the educational message, or both are sent to the mobile device in response to the determined current adherence score not satisfying an adherence threshold.

130. The system of claim 122, wherein the control system is further configured to execute the machine-readable instructions to determine the current adherence score by determining whether a total amount of the substance delivered by a time of day when the current adherence score is determined exceeds a scheduled amount of the substance to be delivered according to the substance replacement therapy.

131. (canceled)

132. The system of claim 122, wherein the control system is further configured to execute the machine-readable instructions to increase or decrease an amount of the substance delivered to the user based on (i) a determination that the current adherence score is different than a previous adherence score, (ii) the current adherence score not satisfying an adherence threshold, or (iii) both.

133-135. (canceled)
